(19) United States
(12) Patent Application Publication
(10) Pub. No.: US 2021/0128496 A1
(43) Pub. Date: May 6, 2021

(54) DROPROPIZINE IN COMBINATION WITH AMBROXOL IN THE DOSAGE FORM OF SYRUP OR TABLETS

(71) Applicant: Productos Farmaceuticos, S.A. DE C.V., Ciudad De Mexico (MX)

(72) Inventor: Jorge Luis Espino Vazquez, Ciudad De Mexico (MX)

(73) Assignee: Productos Farmaceuticos, S.A. DE C.V.s, Ciudad De Mexico (MX)

(21) Appl. No.: 16/497,269

(22) PCT Filed: Apr. 19, 2018
(86) PCT No.: PCT/IB2018/052699

§ 371 Date: Sep. 25, 2019

(30) Foreign Application Priority Data
Nov. 16, 2017 (MX) MX/A/2017/014725

(51) Int. Cl. A61K 31/136; A61K 31/495; A61K 47/38; A61K 9/00; A61K 9/20; A61P 11/14

(52) U.S. Cl. CPC A61K 31/136; A61K 31/495; A61P 11/14; A61K 9/0095; A61K 9/2018; A61K 47/38

(57) ABSTRACT

The present invention relates to a pharmaceutical combination of ambroxol or a pharmaceutically acceptable salt thereof and dropropizine. Moreover, it refers to a pharmaceutical composition, preferably in a form of oral administration, containing said active ingredients, together with one or more pharmaceutically acceptable additives. The invention also relates to the preparation processes and the processes of use of the pharmaceutical combination or the pharmaceutical composition of the present invention.

### CROSS-REFERENCE TO RELATED APPLICATION

This application is a national stage application filed under 35 USC 371 of PCT/IB2018/052699, filed Apr. 19, 2018, which claims the benefit of Mexican Patent Application No. MX/a/2017/014725, filed Nov. 16, 2017, each of which is incorporated herein, in its entirety, by reference.

### TECHNICAL FIELD OF THE INVENTION

The present invention is related to the field of medicaments, preferably for oral administration, intended for the inhibitory and/or suppressive treatment of coughs of any etiology and, at the same time, to keep the airways free of secretions in bronchopulmonary processes, where the viscosity and adherence of the mucus increases.

### BACKGROUND OF THE INVENTION

Cough is a mechanism for the protection of the airways and also the most common respiratory symptom in children and adults. It can originate from innumerable infectious and non-infectious causes, be characterized as productive or dry (non-productive), and be classified, according to duration, into acute (less than 3 weeks), subacute (3-8 weeks) or chronic (more than 8 weeks).

When the cough is not productive and the secretion is very viscous or is very attached to the lower part of the respiratory tract, measures must be taken to make it productive. The cough should be avoided when it is not productive or when its intensity is a problem for the rest of the person or may cause complications.

The compound known under the generic name of dropropizine was disclosed as an active ingredient in Belgian patent application number BE 601,394, which was filed on Mar. 16, 1961. Its chemical formula is C₁₃H₂₀H₂₀O₂ and, according to the IUPAC nomenclature, dropropizine corresponds to the name compound (R, S)-3-(4-phenylpiperazin-1-yl) propane-1,2-diol, and is represented by following structural formula (I):

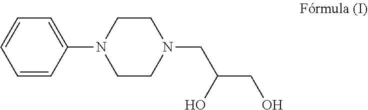 [SMILES: OCC(O)CN1CCN(c2ccccc2)CC1]

This active substance is a synthetic substance that has been found to have an effective antitussive action, it acts as a sedative of peripheral action of cough, not narcotic and, unlike codeine-type sedatives, it lacks depressant action of the central nervous system (CNS).

It exists as a racemic mixture of levo- and dextro-rotary enantiomers. Studies with levodropropizine in animals show that it inhibits the direct stimulation of afferent fibers C of the vagus nerve, with the release of neuropeptides acting on these nerve fibers and participate in the development of the cough reflex.

Likewise, it has been shown that the antitussive activity of dropropizine and its levo enantiomer is comparable; however, racemic dropropizine exhibits a higher CNS depressant effect at the doses studied.

The CNS depressant effects of dropropizine may be related to its affinity for H1, alpha-adrenergic, serotonergic and dopaminergic receptors (Giani R. et al. Synthesis and pharmacological screening of new phenylpiperazinepropane derivatives and their enantiomers.” Arzeimittelforschung. 1988; 38 (8): 1139-41); Noel P R B. Dropropizine (UCB 1967), an Antitussive: Oral Toxicity Study in pure bread dogs. Arzeimittelforschung. 1969; 19: 1246-49; Gatii G., Barzaghi N. et al. Enantioselective effects of levodropropizine and dropropizine on psychomotor functions in normal volunteers: a placebo-controlled, double-blind comparative study. Drugs Exp Clin Res. 1993; 19 (1): 33-9; Banderali G. et al. Efficacy and tolerability of levodropropizine and dropropizine in children with non-productive cough. J Int Med Res. 1995; 23(3):175-83; Kotlar W. et al. A double-blind, cross-over study of a new antitussive agent versus dropropizine and placebo. Geriatric Hospital. Havre. Belgium. Pp. 121-129).

The dropropizine exhibits considerably less CNS depressant effects than codeine, but the antitussive activity between both is comparable. When secondary insomnia to cough occurs in patients, the dropropizine is superior to levodropropizine for its greater sedative effect. The racemic dropropizine has been marketed for the symptomatic treatment of cough for many years. However, very little information is available on its pharmacokinetics as a racemate.

The dropropizine is absorbed from the gastrointestinal tract and distributed throughout the body, its elimination half-life is hours, while cough suppression is reached 1 hour after the administration of the drug and the effect persists for a period of 6 hours. The appearance of the antitussive effect and its duration is similar for both dropropizine and levodropropizine (Fumagalli G. et. Al. A comparative study of the antitussive activity of levodropropizine and dropropizine in the citric acid-induced cough model in normal subjects. Drugs Exp Clin Res. 1992; 18 (7): 303-09).

Particularly, levodropropizine exhibits a similar pharmacokinetic behavior (absorption, distribution, metabolism, and elimination) in animals and in man, it is estimated that the oral bioavailability of levodropropizine is greater than 75% and that its plasma protein binding is of around 11-14%. After oral administration, the Tₘₐₓ is reached about 0.6 to 1 hour. Plasma concentrations decline mono-exponentially once Cₘₐₓ is reached and can be detected in plasma up to 10 hours after administration and has a half-life t_{1/2}=2 hours (Yan L. et al. HPLC Determination and steady-state Bioavailability Study of Levodropropizine sustained-release tablets in dogs. Arch Pharm Res. 2006; 29 (6): 514-519; Jeon S., Lee J., Houg T., et al. Pharmacokinetics and Safety of Levodropropizine Controlled Release Tablet after Repeated Dosing in Healthy Male Volunteers. J Korean Soc Clin Pharmacol Ther. 2013; 21 (2): 113-119; Jang J., Seo-Ji., Jo Min-Ho., Et. al.

Relative bioavailability of levodropropizine 60 mg capsule and syrup formulations in healthy male Korean volunteers: a single dose, randomized-sequence, open-label, two-way crossover study. Int. Journal of Clinical Pharmacology and Therapeutics. 2013; 6 (2): 152-160).

The dropropizine has no depressive effect on respiratory function or airway clearance mechanisms. Neither does it modify the rheological properties of mucus or the ciliary activity of the bronchial epithelium. It is free of effects at the cardiovascular level and its use does not induce physical dependence. It is indicated as a non-narcotic peripheral cough suppressant, in the treatment of cough of any etiology, even in the presence of bronchospasms and respiratory insufficiency; pharyngitis, laryngitis, acute or chronic tracheitis, irritative cough, and general conditions.

Generally, it is administered orally and rectally, in the dosage forms of syrup, tablet or suppository. In adults and over years old, oral administration in the form of syrup and tablets is 30 mg of dropropizine every 8 hours and, in children, the dose varies from 7.5 mg to 15 mg every 8 hours, in the form of syrup. The dose rectally is 20 mg every 8 hours.

However, adverse effects on the gastrointestinal tract have been observed in treatment with dropropizine, for example nausea and vomiting; in addition, other effects such as headache, drowsiness and allergic reactions, may also occur.

In addition to the foregoing, the present invention is also directed to solve the problem of keeping the airways free of secretions in bronchopulmonary processes, where the viscosity and adhesion of mucus increases.

It is known that the secretion of the respiratory tract along with the ciliary component, constitute the most important mucosal protection system against infectious agents, suspended particles in inspired air, and extreme moisture and temperature variations. The mucus traps the particles and purges by a coordinated process between the cilia, which beat rhythmically, and the layer of mucus.

The secretion is basically due to the mucus and serous glands of the submucosa, and to the goblet cells of the mucosa. The secretion of the submucosal glands is influenced by nerve, chemical and mechanical stimuli, while that of goblet cells does not respond to nerve stimuli. The main constituents of this secretion form a complex mixture, composed mostly of water (95%), acid glycoproteins (2%), lipids (0.5-1%) and other proteins in lower proportion.

It has been discovered that these molecules aggregate and cross-linked to form a three-dimensional matrix by various forces: hydrogen bonds, ionic bonds and covalent bonds. Hydrogen bonds (weak) determine the viscous properties; the ionic and covalent bonds (stronger) determine elasticity and viscosity. Thus, the more acid is the secretion, the greater the viscoelasticity.

The compound known under the name of ambroxol was disclosed on Apr. 26, 1968, as an active ingredient in the French patent application number FR 1,522,709 dated May 10, 1967. The chemical formula is C₁₃H₁₈Br₂N₂₀, its IUPAC chemical name is trans-4-[(2-amino-3,5-dibromophenyl) methylamino] cyclohexane-1-ol and is represented by Formula (II):

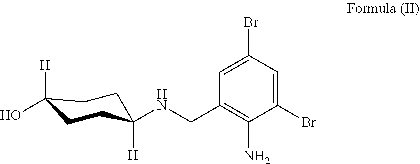

This molecule acts intracellularly by promoting the synthesis and secretion of alveolar and bronchial surfactants, forming a film throughout the entire respiratory epithelium. In addition, it has a mucolytic-expectorant action and increases the vibratory power of the ciliary epithelium. All these modifications reduce the mucus adhesiveness, facilitating the sliding and transport of bronchial secretions to the outside avoiding concomitant obstructions.

The absorption of all non-delayed oral dosage forms of ambroxol is rapid and nearly complete, the linearity of the dose is within the therapeutic range. The maximum plasma levels are reached between 0.5 and 3 hours, and after 6.5±2.2 hours for the slow-release formulation. The extended-release capsules show a relative availability of 95% compared to the 30 mg tablets.

At therapeutic doses, the binding to plasma protein is about 90%. The distribution of ambroxol administered orally, from the blood to the tissue is rapid and pronounced, the highest concentration of the active substance has been found in the lungs. Approximately 30% of the orally administered dose is eliminated by the first-pass metabolism. Studies in human liver microsomes show that CYP3A4 is the predominantly responsible isoform of ambroxol metabolism. It is first metabolized in the liver by conjugation. The terminal elimination half-life is 10 hours. The renal clearance represents 8% of total clearance (Wen A. et al. Simultaneous determination of amoxicillin and ambroxol in human plasma by LC-MS/MS: Validation and application to pharmacokinetics study. Journal of Pharmaceutical and Biomedical Analysis, 2008 (48) 829-834; Lee H J et al. Bioequivalence assessment of ambroxol hydrochloride tablets after single oral dose administration to healthy male volunteers. Pharmacological Research. 2004, 49(1):93-98).

The ambroxol is indicated as mucolytic with expectorant and surfactant action, as an adjuvant in bronchopulmonary processes, where the viscosity and adherence of the mucus increases, in processes in which it is necessary to keep the airways free of secretions, such as rhinitis, sinusitis, bronchitis, bronchiectasis and atelectasis due to mucosal obstruction; during the use of tracheostomy, and in the pre and postoperative period of geriatric patients who may develop hypostatic pneumonia.

This active ingredient can be administered orally, being available in the pharmaceutical forms of capsule, syrup, solution, drops and tablets, at a dose of 30 mg every 8 hours in adults and children over 12 years old and, the weight dose is 1.5 to 2 mg/kg/day in children under 12 years old. The age and gender do not affect the pharmacokinetic properties of ambroxol to a relevant clinical degree and no dose adjustment is required.

The ambroxol is generally well tolerated. However, gastrointestinal disorders such as pyrosis, dyspepsia, nausea, vomiting, diarrhea, and abdominal pain have been reported; rash, urticaria, angioedema, anaphylactic reactions (including anaphylactic shock) and other allergic reactions.

In the literature there are several documents that disclose the pharmacology of the drugs dropropizine and ambroxol, but as individual drugs (for example, E. Fatma et al. “Effects of safety warnings on prescription rates of cough and cold medicines in children below 2 years of age” (2011), Br. J. Clin Pharmacol. 71:6, 943-950; o Aracy Pereira Silveira Balbani “Cough: neurophysiology, methods of research, pharmacological therapy and phonoaudiology”. Int. Arch. Otorhinolaryngol. 2012; 16(2):259-268).

The international patent application with publication number WO 2013/154347 published on Oct. 17, 2013, discloses an oral liquid formulation comprising the combination of active ingredients ambroxol and levodropropizine, together with a buffering agent, among other pharmaceutically acceptable ingredients. This application reports that ambroxol and levodropropizine have been used separately in preparations in the form of syrups. However, this document does not describe a combination of ambroxol and dropropizine, nor a formulation containing said combination of active ingredients.

Additionally, WO 2013/154347 reports that, when ambroxol and levodropropizine are in a liquid formulation together, an increase in the number of related substances to these active ingredients has been observed, so it was attempted to remedy this problem using a buffering agent that controls the pH.

Accordingly, it is expected that when ambroxol and dropropizine are in the same pharmaceutical form, the problem of the production of ambroxol-related substances is also present, because, on the one hand, the racemic mixture includes levodropropizine and, on the other hand, that once the formulation is administered, the active ingredients are at the pH of the stomach ranging from about 1 to about 3, wherein undesired impurities related to ambroxol are produced.

Moreover, it is well known that various factors influence the absorption of an oral administration pharmaceutical form (the pH, the amount and type of food, drug solubility). But there are also other characteristics of the subject (for example, the absorption surface, the rate of intestinal transit, as well as some pathological processes), which can substantially modify the absorption process.

In addition, some drugs can be mucosal, causing adverse effects and the consequent therapeutic noncompliance. On the other hand, many orally administered drugs suffer from important hepatic metabolism (First Pass Effect), which substantially limits their administration by this route.

In accordance with the foregoing, despite all efforts, there has persisted the need to find alternatives to stable pharmaceutical compositions which can preferably be administered via de oral administration route and which simultaneously provide a mucolytic-expectorant activity, a peripheral cough suppressant activity and also exhibiting lower adverse effects.

### BRIEF DESCRIPTION OF THE INVENTION

As a result of extensive research and development, the inventor of the present patent application has experimentally and unexpectedly found that a pharmaceutical combination of dropropizine and ambroxol, and/or a pharmaceutical composition, preferably suitable for oral administration, containing as active ingredients dropropizine and ambroxol or a pharmaceutically acceptable salt thereof, provides a mucolytic-expectorant and peripheral antitussive activity, it is also stable either in liquid and solid pharmaceutical forms and does not have the adverse effects of dropropizine, nor the adverse effects that may be derived from ambroxol impurities produced when ambroxol and levodropropizine of the racemic mixture are in the same pharmaceutical composition.

The pharmaceutical combination of dropropizine and ambroxol or a pharmaceutically acceptable salt thereof, as well as a pharmaceutical composition comprising said active ingredients according to the present invention, has the additional advantage that it can be adapted to be administered by the oral route in a liquid or solid pharmaceutical form, which makes it possible to improve the patient treatment compliance, as simple and easy to use forms and provide a metered dose of active ingredients in a comfortable portable container.

In a first aspect, the present invention consists of a pharmaceutical combination containing as active ingredients: ambroxol or a pharmaceutically acceptable salt thereof and dropropizine.

It also refers in one embodiment to a pharmaceutical composition, preferably in a form of oral administration, which contains as active ingredients: ambroxol or a pharmaceutically acceptable salt thereof and dropropizine, and one or more pharmaceutically acceptable additives.

In a second aspect, the present invention consists in a process of preparing the pharmaceutical composition in an oral administration form containing as active ingredients: ambroxol or a pharmaceutically acceptable salt thereof and dropropizine.

In a third aspect, the invention relates to a pharmaceutical combination of ambroxol or a pharmaceutically acceptable salt thereof and dropropizine. It also refers to a pharmaceutical composition, preferably for oral administration, containing ambroxol or a pharmaceutically acceptable salt thereof and dropropizine, for use in the inhibitory and/or cough suppressant treatment of any etiology, even in the presence of bronchospasms and respiratory insufficiency; pharyngitis, laryngitis, acute or chronic tracheitis, irritative cough; and as a mucolytic-expectorant action and surfactant, as an adjuvant in bronchopulmonary processes, when the viscosity and adherence of the mucus increases, in the processes in which it is necessary to keep the airways free of secretions such as rhinitis, sinusitis, bronchitis, bronchiectasis and atelectasis due to mucus obstruction; during the use of tracheotomies, and in the pre and postoperative period of geriatric patients who may develop hypostatic pneumonia; said pharmaceutical composition contains as active ingredients: ambroxol or a pharmaceutically acceptable salt thereof and dropropizine.

### DETAILED DESCRIPTION OF THE INVENTION

In the first aspect, the present invention consists of a pharmaceutical combination of ambroxol or a pharmaceutically acceptable salt thereof and dropropizine.

Also, in one embodiment of the invention, it consists of a pharmaceutical composition, preferably in a form of oral administration, which contains as active ingredients: ambroxol or a pharmaceutically acceptable salt thereof and dropropizine, and one or more pharmaceutically acceptable additives.

Preferably, the pharmaceutically acceptable salt of ambroxol is ambroxol hydrochloride.

The pharmaceutical form of oral administration according to the pharmaceutical composition of the present invention may be chosen from solid forms and liquid forms.

Some advantages of solid oral administration forms are that they can solve incompatibility problems, mask unpleasant flavors and even regulate the release of active ingredients.

The pharmaceutical forms of solid oral administration according to the pharmaceutical composition of the invention can be particularly selected from the group comprising: uncoated tablets, which are obtained by simple compression and are composed of the active substances dropropizine and ambroxol together with one or more additives, such as diluents, binders, disintegrants, lubricants; multi-layered tablets, which are obtained by multiple compressions whereby several superimposed cores are obtained, with different compaction in each of them; coated tablets or dragees, wherein the coating may be of sugar or of a polymer that breaks when it reaches the stomach; gastro-resistant or enteric coated tablets, which resist acid secretions from the stomach, finally breaking down in the small intestine; controlled-release tablets, which control the release of the active substance in the body; and effervescent tablets, which are obtained by compression of a granule of effervescent salts, generally an acid (citric acid) and an alkali (sodium bicarbonate), which, in contact with water, originate carbon dioxide which decomposes the mass of the tablet and releases the active ingredients of the invention.

The preferred pharmaceutical forms of solid oral administration according to the present invention are among other powders, granules, tablets (effervescent, chewable, dragees), capsules (hard, soft or beads, gastro-resistant or modified-release coating), seals, pills, tablets, and official lozenges.

The most preferred pharmaceutical form of solid oral administration according to the present invention is the tablet form.

Moreover, the oral administration pharmaceutical form of the pharmaceutical composition of the invention can be selected in an immediate release or modified release form.

Examples of modified release forms are delayed release systems, which are designed to save gastric pH or to prevent gastrolesivity of any or both drugs; pulsatile delayed release systems, which are sequential drug release; extended-release systems, designed to prolong the plasma concentration of one or both drugs, to improve the pharmacokinetic characteristics of the drug or to reduce fluctuations in plasma levels; or floating and bioadhesives designed to increase the gastric residence period.

When the pharmaceutical composition of the present invention is in a solid oral administration form, the content per unit dose of dropropizine is in the range of about 12.73% w/w to about 14.73% w/w, preferably in the range of about 13.22% w/w to about 14.18% w/w and more preferably at about 13.7% w/w. On the other hand, the content per unit dose of ambroxol hydrochloride is in the range of between about 12.74% w/w to about 14.73% w/w, preferably in the range of about 13.22% w/w to about 14.18% w/w and more preferably about 13.7% w/w.

The one or more pharmaceutically acceptable additives contained in the pharmaceutical composition of the present invention are selected according to the pharmaceutical form of chosen solid oral administration.

For the purposes of this patent application, the term “additives” refers to any substance that is included in the formulation of the drugs and acts as a vehicle, conservative or modifier of any of its characteristics to promote its effectiveness, safety, stability, appearance or acceptability. This term interchangeably includes pharmaceutically acceptable excipients, carriers or carriers that may be contained in the pharmaceutical composition of the present invention.

In the event that the form of the pharmaceutical composition of the present invention is a tablet, the one or more pharmaceutically acceptable additives may preferably be selected from the group consisting of microcrystalline cellulose, lactose, croscarmellose sodium, colloidal silicon dioxide and/or magnesium stearate.

The content per unit dose of additives, when the pharmaceutical composition of the present invention is in a solid oral administration form, it is in the range of about 70.53% w/w to about 74.52% w/w, preferably in the range of about 71.52% w/w to about 73.52% w/w and more preferably at about 72.52% w/w.

On the other hand, liquid oral administration forms are generally chosen because they have the advantages that they do not pose disintegration or dissolution problems in the digestive tract, which condition a faster therapeutic action. However, they are not protected in case of reactivity against digestive juices.

They are often of choice, particularly in children or adults unable to swallow tablets or capsules.

The additives used in the liquid oral forms may be aqueous, mucilage or hydroalcoholic. The aqueous additives serve to dissolve water-soluble active ingredients. The most common are syrups (which contain a high concentration of sugar, up to 64% by weight). The mucilages are viscous liquids resulting from the dispersion of gummy substances (gum arabic, tragacanth, agar, methylcellulose) in water, these vehicles are useful for preparing suspensions and emulsions. The hydroalcoholic vehicles or elixirs are hydroalcoholic solutions (25% alcohol) sweeteners used to dissolve water-soluble substances and alcohol.

The preferred pharmaceutical forms of liquid oral administration according to the pharmaceutical composition of the invention are syrups (solution), elixirs (solution), suspensions, extemporaneous suspension (that which is prepared at the time of administration), drops and emulsions.

The most preferred form of liquid oral administration according to the pharmaceutical composition of the invention is the syrup form.

When the pharmaceutical composition of the present invention is in a form of liquid oral administration, the content per unit dose of dropropizine is in the range of between about 0.27% w/v or about 0.33% w/v, preferably in the range of between about 0.29% w/v or about 0.31% w/v, and more preferably at about 0.30% w/v. On the other hand, the content per unit dose of ambroxol hydrochloride is in the range of about 0.27% w/v to about 0.33% w/v, preferably in the range of about 0.29% w/v to about 0.31% w/v, and more preferably about 0.30% w/v.

The one or more pharmaceutically acceptable additives contained in the pharmaceutical composition of the present invention are selected according to the pharmaceutical form of chosen liquid oral administration.

The liquid oral administration forms of the present invention may contain auxiliary substances for the preservation, stability or masking of the taste of the pharmaceutical preparation (preservatives, antimicrobials, antioxidants, buffers, solubilizers, stabilizers, flavorings, sweeteners and authorized colorants).

In one embodiment, in case the liquid oral administration form is a syrup, the one or more pharmaceutically acceptable additives may preferably be selected from the group consisting of neohesperidine dihydrochalcone, sucrose, glycerol, benzoic acid, citric acid, sodium metabisulfite, propylene glycol, ethyl alcohol, flavorings and/or purified water.

When the pharmaceutical composition of the present invention is in a form of liquid oral administration, the content per unit dose of additives is in the range of about 43.8% w/v or about 48.42% w/v, preferably in the range of about 44.96% w/v to about 47.26% w/v and more preferably at about 46.11% w/v.

Furthermore, when the pharmaceutical composition of the present invention is in a form of liquid oral administration, the pH of the composition should preferably be in the range of 4.0 to 5.5, in another embodiment in the range of 4.0 to 5.0, in another additional embodiment in the range of 4.0 to 4.4 and in another embodiment in the range of 4.5 to 4.7.

The second aspect of the present invention relates to a process for preparing the pharmaceutical composition of the invention, preferably in a form of oral administration, which contains as active ingredients: ambroxol or a pharmaceutically acceptable salt thereof and dropropizine.

Preferably, the pharmaceutically acceptable salt of ambroxol is ambroxol hydrochloride.

The particular process for preparing the pharmaceutical composition of the present invention is selected according to the chosen oral dosage pharmaceutical form.

If the oral administration form of the pharmaceutical composition of the invention is a tablet, the process according to the second aspect of the invention can be carried out basically by three methods, namely: direct compression, dry compression (dry granulation) and wet compression (wet granulation).

Preferably the process for preparing the tablets of the present invention is direct compression. This process involves the step of directly compressing the mixture of the drugs ambroxol and dropropizine, together with the additives, which are safe substances that allow fluidity and increase cohesiveness, to shape the tablet, they must also have compressibility properties.

More particularly, the process for preparing the pharmaceutical composition of the present invention begins with the dispensing of raw materials. The production begins with the mixture of raw materials, which are added to container preferably of stainless steel, finishing the addition the container is closed and placed in a mill. The final particle size is controlled during the process. The bulk powder is received in another container for example also of stainless steel, said container being translated for the next operation where mixing is performed. After the mixture time, the container is weighed with the bulk powder and the yield is calculated. Prior to tableting, the critical parameters are adjusted: Filling height, Pre-compression height, Compression height, and adjustment parameters: Pre-compression force, Compression force, Production speed, Feeder speed 1 and Feeder speed 2. Once the adjustment is done, the tableting process begins, during which the corresponding sampling is carried out for chemical analysis and for the controls in the process: Weight, hardness, friability and disintegration time.

In another embodiment of the second aspect of the present invention, the oral administration form of the oral pharmaceutical composition of the present invention is in the syrup form. The corresponding preparation process is preferably carried out by the hot dissolution of the sugar.

The process for preparing an oral pharmaceutical composition of the present invention in syrup form broadly involves the dissolution and sequential mixing of the active ingredients with pharmaceutically acceptable additives.

Specifically, the syrup preparation of the present invention is carried out by mixing the sweetener, for example neohesperidine-dihydrochalcone in purified water under constant agitation. The sucrose is then added and heating at an elevated temperature starts, for example, between about 80° C. to 85° C. At the end of the heating time, the solution is cooled, for example, to a temperature below about 40° C. The other pharmaceutically acceptable additives may then be dissolved and/or added, such as glycerol, alcohol, benzoic acid, and sodium metabisulfite, while maintaining constant stirring for a while.

The dropropizine is first added in purified water, under constant agitation at elevated temperature, for example, between and 81° C., until total dissolution. In the same manner, ambroxol or ambroxol hydrochloride can be added in purified water by keeping stirring until completely dissolved. These solutions are subsequently integrated into the syrup mixture, the pH of the syrup is adjusted by cooling the sample, for example, between about 19 and 21° C. The pH of the syrup should preferably have an approximate pH of 4.5 and 4.7 if not, it can be adjusted for example with a solution of citric acid, while maintaining constant agitation. Finally, the syrup with all the ingredients is diluted and filtered at room temperature.

In the third aspect, the invention relates to a pharmaceutical combination and/or a pharmaceutical composition, preferably in an oral pharmaceutical form, for use in the inhibitory and/or cough suppressant treatment of any etiology, even in the presence of bronchospasms and respiratory insufficiency; pharyngitis, laryngitis, acute or chronic tracheitis, irritative cough; and/or as a mucolytic-expectorant action and surfactant, as an adjuvant in bronchopulmonary processes, where the viscosity and adherence of mucus increases, in the processes in which it is necessary to keep the airways free of secretions such as rhinitis, sinusitis, bronchitis, bronchiectasis and atelectasis due to mucus obstruction; during the use of tracheotomies, and in the pre and postoperative period of geriatric patients who may develop hypostatic pneumonia; wherein the pharmaceutical combination and/or the pharmaceutical composition contain as active ingredients: ambroxol or a pharmaceutically acceptable salt thereof and dropropizine. Preferably, wherein the pharmaceutically acceptable salt of ambroxol is ambroxol hydrochloride.

More particularly, the invention relates to a pharmaceutical combination and/or a pharmaceutical composition, preferably for oral administration, for use in the treatment of diseases of the respiratory system that concur simultaneously with irritative and productive cough and wherein they are indicated as concomitant the inhibition of the cough reflex and a mucolytic and expectorant effect; as secretolytic therapy in acute and chronic bronchopulmonary diseases associated with abnormal mucus secretion and impaired mucosal transport and as a non-narcotic peripheral cough suppressant; indicated in the treatment of cough of any etiology, even in the presence of bronchospasms and respiratory insufficiency; pharyngitis, laryngitis, acute or chronic tracheitis, irritative cough, wherein said pharmaceutical combination and/or said pharmaceutical composition contains as active ingredients ambroxol or a pharmaceutically acceptable salt thereof and dropropizine. Preferably, the pharmaceutically acceptable salt of ambroxol is ambroxol hydrochloride.

In another embodiment, the pharmaceutical combination and/or the pharmaceutical composition of the present invention is for use in the treatment of diseases of the respiratory system that concur simultaneously with irritative and productive cough and wherein concomitant inhibition of the cough reflex and a mucolytic-expectorant effect. Said pharmaceutical combination and/or said pharmaceutical composition contains as active ingredients ambroxol or a pharmaceutically acceptable salt thereof and dropropizine. Preferably, the pharmaceutically acceptable salt of ambroxol is ambroxol hydrochloride.

Also, the pharmaceutical combination and/or pharmaceutical composition of the present invention is for use in the treatment of diseases of the respiratory system that concur simultaneously with irritative and productive cough and wherein concomitantly indicated the inhibition of the cough reflex and an effect mucolytic and expectorant, in the group of patients presenting insomnia secondary to cough. Said pharmaceutical combination and/or said pharmaceutical composition contains as active ingredients ambroxol or a pharmaceutically acceptable salt thereof and dropropizine. Preferably, the pharmaceutically acceptable salt of ambroxol is ambroxol hydrochloride.

In the following examples described below, two pharmaceutical compositions according to the present invention are shown, the first in a solid oral administration form (a tablet) and the second in a liquid oral administration form (a syrup). A comparative pharmacokinetic test among three pharmaceutical preparations is also shown: a solution of ambroxol of 300 mg/100 mL, dropropizine syrup of 300 mg/100 mL, and the fixed combination of dropropizine-ambroxol syrup of 300-300 mg/100 mL, all of which exemplify the best mode or the best manner provided by the applicant to make or put into practice the claimed invention, as well as the industrial application of the invention.

### EXAMPLES

### Example 1. Tablets Containing 30 mg of dropropizine and 30 Mg of Ambroxol Hydrochloride

An oral pharmaceutical composition in the form of a tablet containing dropropizine and ambroxol hydrochloride as active ingredients is shown below.

Each tablet contains:

| | Amount | Percentage used |
|---|---|---|
| Ingredient | (mg) | (% w/w) |
| Dropropizine | 30.000 | 13.761 |
| Ambroxol | 30.000 | 13.761 |
| Hydrochloride | | |
| Microcrystalline | 42.800 | 19.633 |
| cellulose | | |
| Lactose monohydrate | 12.600 | 5.780 |
| (mesh 200) | | |
| Lactose monohydrate | 93.600 | 42.936 |
| D.C. (Spray Dried) | | |
| Croscarmellose sodium | 5.000 | 2.294 |
| Colloidal silicon | 2.200 | 1.009 |
| dioxide U-200 | | |
| Magnesium stearate | 1.800 | 0.826 |

### DESCRIPTION OF THE MANUFACTURE PROCESS

The oral pharmaceutical composition in the form tablet of the invention consists of a product with a fixed-dose combination that can be manufactured by direct compression. For this manufacturing method, the additives or excipients are usually powders for direct compression, sometimes spherical, integrated by associations that guarantee adequate flow and suitable compacting properties.

Stage of Bulk Powder Production

The process begins with the dispensing of raw materials. In the mixing area, production begins with the mixture, under the principle of diffusion mixing operation (Difusion Mixers), of the raw materials, which are added to a stainless steel container in the following order: Lactose monohydrate D.C. “Spray Dried” (42.936%), lactose monohydrate mesh 200 (5.780%), dropropizine (13.761%), colloidal silicon dioxide U-200 (1.009%), ambroxol hydrochloride (13.761%), croscarmellose sodium (2.294%) and microcrystalline cellulose pH 102 (19.633%). Once the addition is finished, the container is closed and a mill sieve is performed (screening mill) with a mesh of about 850 μm. The screening is performed at a speed of about 2000 rpm of the mill and about 50 rpm of the worm, in order to control the final particle size. The bulk powder is received in another container preferably of stainless steel, said container being translated for the next operation where it is mixed for about 7 minutes at about 9 rpm.

At the end of the mixing time, the magnesium stearate is added, which can be passed through a No. 20 sieve, and final mixing is performed to incorporate the lubricant, mixing for about 2 minutes at about 9 rpm. After the mixing time, the container is weighed with the bulk powder and the yield is obtained.

### Stage of Bulk Tablets Production

The production process of dropropizine-ambroxol Bulk Tablets is performed in a tableting area, starting the process with the feeding of the powder by opening the discharge valve and starting with the adjustment of the tableting machine, according to the critical parameters: Filling height, Pre-compression height, Compression height and adjustment parameters: Pre-compression force, Compression force, Production speed, Feeder speed 1 and Feeder speed 2.

Once the adjustment has been made, the tableting process begins, where the tablets are received in wedges with a double polythene bag. During the tableting process, the corresponding sampling for chemical analysis and for the controls in process are performed: Weight, hardness, friability and disintegration time.

### Example 2. Syrup Containing 7.5 mg Dropropizine and 7.5 mg of Ambroxol

An oral pharmaceutical composition in the form of syrup containing dropropizine and ambroxol hydrochloride as active ingredients is described below.

Each 2.5 mL of syrup contains:

| | | |
|---|---|---|
| | | Percentage used |
| Ingredient | Amount | (%) |

| | | | | |
|---|---|---|---|---|
| DL-Dropropizine | 7.500 | mg | 0.300 | w/v |
| Ambroxol Hydrochloride | 7.500 | mg | 0.300 | w/v |
| Neohesperidine | 0.125 | mg | 0.005 | w/v |
| Dihydrochalcone | | | | |
| Sucrose | 1000.000 | mg | 40.000 | w/v |
| Glycerol | 125.000 | mg | 5.000 | w/v |
| Benzoic acid | 2.250 | mg | 0.090 | w/v |
| Citric Acid Anhydrous* | q.s. | mg | q.s. | w/v |
| Sodium Metabisulfite | 0.500 | mg | 0.020 | w/v |
| Propylene Glycol | 25.000 | mg | 1.000 | m/v |
| Ethanol 96° G.L. | 0.025 | mL | 1.000 | v/v |
| Tangerine Flavor | 0.005 | mL | 0.200 | v/v |
| Purified Water | 2.500 | mL | 100.00 | v/v |

Quantum sufficit for pH adjustment between 4.0 to 5.5, preferably between 4.0 and 5.0.

### Stage of Bulk Syrup Production

The production process of dropropizine-ambroxol syrup begins with the dispensing of raw materials. Subsequently, the sugar syrup is prepared, by loading about 3000 L of purified water to a stainless-steel tank, the stirring speed is adjusted between about 30 and 50 Hz; 0.005% w/v of neohesperidine-dihydrochalcone is added, stirring for a period between about 10 to 12 minutes.

With continuous agitation between about 30 and 50 Hz, 40.000% w/v of sucrose is added to the manufacturing tank solution (for example, 6000 L), after the addition heating is started at a temperature between about 80° C. at 85° C. Reached the temperature, it is maintained between about 20 and 30 minutes. Upon completion of the heating time, the solution of the manufacturing tank (for example, 6000 L) is cooled to a temperature below 40° C.

Maintaining the agitation speed between about 30 and 50 Hz in the manufacturing tank (for example, 6000 L) 5.000% w/v glycerol is added and stirred for about 10 to 15 minutes. In a dissolution tank for example of stainless steel, 1.000% v/v of ethyl alcohol is placed, and 0.090% w/v of benzoic acid is added, stirred at a speed between about 30 and 50 Hz until completely dissolved. Subsequently, the benzoic acid solution is transferred to the manufacturing tank (for example, 6000 L) where stirring is maintained at a speed between about 30 and 50 Hz and stirred between about 10 and 12 min.

In a solution tank preferably made of stainless steel, 100.00% v/v of purified water is added, the agitation speed is adjusted between about 30 and 50 Hz at a temperature between about 80 and 81° C., subsequently 0.300% w/v of dropropizine is added by stirring until completely dissolved.

After the dissolution of the dropropizine, this solution is transferred to a manufacturing tank (for example, 6000 L) where stirring is maintained between about 30 and 50 Hz for about 15 and 20 minutes. After the stirring time is over, the syrup pH is adjusted by cooling the sample between about 19 and 21° C. The pH of the syrup should be in the range of 4.0 to 5.5 and, preferably a pH between about 4.5 and 4.7, if not, it can be adjusted with a 10% w/v citric acid solution, maintaining agitation between about 30 and 50 Hz during adjustment.

Subsequently in a dissolution tank for example of stainless steel about 1000 liters of water is added, stirring is started between about 30 and 50 Hz, 0.300 w/v of ambroxol hydrochloride is added by maintaining the stirring until complete dissolution.

The ambroxol hydrochloride solution is transferred to the manufacturing tank (for example, 6000 L) by using a transfer pump, After the transfer, stirring is maintained between about 30 and 50 Hz for about 10 to 15 minutes.

In another dissolution tank, about 80 L of purified water is added by adjusting the stirring between about 30 and 50 Hz, Subsequently, 0.020 w/v sodium metabisulfite is added while stirring until completely dissolved. The sodium metabisulfite solution is transferred to the manufacturing tank (for example, 6000 L), stirring between about 30 and 50 Hz for about 10 to 15 min. At this point it is verified if there is a pH in the range of 4.0 to 5.5 and, preferably a pH between about 4.5 and 4.7, if necessary, it can be adjusted with a 10% w/v citric acid solution.

Once the pH is verified, it is added to the manufacturing tank (for example, 6000 L), 1.000% w/v of propylene glycol, maintaining the stirring between about 30 and 50 Hz, stirring for 10 to 15 minutes. Subsequently, 0.200 v/v of flavoring can be added and stirred for about 10 to 15 minutes.

The stirring of the manufacturing tank is stopped and a volume of for example 6000 L is started. Then, stirring is resumed at a speed between about 30 and 50 Hz between about 10 and 15 minutes.

After stirring time, the solution is filtered through a filter of for example 5 microns (polypropylene), checking that the filter pressure is between about 1.1 to 1.4 kg/cm².

The bulk is kept in the manufacturing tank closed at room temperature, until its release.

### Example 3. Comparative Pharmacokinetic Test

An open study was conducted, Williams design, 3×6×3 cross, randomized, single-dose, in three treatments, three periods and six sequences under fasting conditions, in a healthy Mexican population. For this, 42 healthy research subjects of both sexes participated. The chronological age fluctuated between 18 and 55 years, with a BMI (Body Mass Index) between 18.0 to 27.0 kg/m².

The estimated sample size of 42 subjects contemplated an 18% drop-out necessary for the study. It was assumed that the distribution of the response variable is Normal and that the statistical test to reject the null hypothesis was the unilateral non-inferiority t-Student test for two related samples. It was considered that, if the response variable did not meet the Normality requirement, it would be necessary for the sample size to be large enough for the results to be good approximations (N>30).

This comparative pharmacokinetic test was performed with three pharmaceutical preparations: a 300 mg/100 mL ambroxol solution (0.3% w/v ambroxol), a 300 mg/100 mL dropropizine syrup (0.3% w/v dropropizine) according to Example 2 but without ambroxol, and a fixed combination of dropropizine-ambroxol in the form of syrup of 300-300 mg/100 mL (0.3% w/v of dropropizine and 0.3% w/v of ambroxol), according to the syrup formulation of Example 2.

By statistical methods, the similarity between the pharmacokinetic parameters (ABC₀₋₁, ABC_{0-∞}, C_{máx}, T_{máx}, Vd/F, Cl/F, T_{1/2}) was determined when administering the medication in fixed combination compared to the administration of Medicines individually. In addition to the information obtained, the bioavailability of the components of the formulations was compared and the non-interaction of the medications was evaluated.

The clinical stage of the study consisted of three phases: pre-experimental (selection of research subjects), experimental (three periods), and post-experimental (high research subjects). After the completion of the clinical stage, the analytical stage was passed.

For the administration of the medication, the zero-time sampling was obtained after a previous fast of at least 10 hours, then 10 mL of a solution/syrup of 300 mg/100 mL (equivalent to 30 mg of ambroxol and/or dropropizine) of the corresponding medications according to prior randomization, orally. In all cases, the medication was administered with a volume of water of 250 mL at room temperature.

The study concluded in a timely manner with 41 volunteers (19 females and 22 males) who met the inclusion criteria and none of the established exclusion criteria. All participating volunteers were duly informed of the nature, scope, and risks of the study and freely gave their informed consent in writing.

During the development of the present study, there were three adverse events with the administration of individual formulation dropropizine; two volunteers had headache and another volunteer had dermatitis, all adverse events were of moderate intensity. At the end of the first week after the last sample of the second period, a clinical interview was carried out following up on possible adverse events of the volunteers, the 41 volunteers were clinically asymptomatic.

However, the fixed combination of dropropizine-ambroxol hydrochloride in the form of syrup of 300-300 mg/100 mL, according to the formulation of Example 2, did not present adverse effects on the research subjects, which is unexpected, since the fixed combination of the present study had to present at least the profile of adverse effects that were observed in this study with the formulation of dropropizine syrup of 300 mg/100 mL.

This study demonstrates that the adverse effects observed when dropropizine is formulated as the only active ingredient disappear when dropropizine is formulated in combination with ambroxol.

Additionally, the 90% confidence intervals of the pharmacokinetic parameters ln (ABC₀₋₁), ln (ABC₀₋₂₈) and ln (C_{máx}) in the comparative bioavailability test fell in the range 80-125%, so it could conclude that the fixed combination consisting of dropropizine and ambroxol hydrochloride, shows an equivalent comparative bioavailability of the drugs that compose it with respect to its components separately.

We claim:

1. A pharmaceutical composition comprising ambroxol or a pharmaceutically acceptable salt thereof and dropropizine.

2. A pharmaceutical composition comprising ambroxol or a pharmaceutically acceptable salt thereof and dropropizine, and one or more pharmaceutically acceptable additives.

3. The pharmaceutical composition according to claim 2, characterized in that it is in an oral administration form.

4. The pharmaceutical composition according to claim 3, wherein pharmaceutically acceptable salt of ambroxol is ambroxol hydrochloride.

5. The composition according to claim 4, wherein the oral administration form is a solid form.

6. The composition according to claim 5, wherein the oral administration form is a tablet.

7. The composition according to claim 6, wherein the content per unit dose of dropropizine is between 12.73% w/w to 14.73% w/w and the content per unit dose of ambroxol hydrochloride is 12.73% w/w to 14.73% w/w.

8. The composition according to claim 7, wherein the one or more pharmaceutically acceptable additives are selected from the group consisting of microcrystalline cellulose, lactose, sodium croscarmellose, colloidal silicon dioxide and/or magnesium stearate.

9. The composition according to claim 3, wherein the oral administration form is a liquid form.

10. The composition according to the preceding claim, wherein the liquid oral administration form is a syrup.

11. The composition according to claim 10, wherein the content per dose unit of dropropizine is between 0.27% w/v and 0.33% w/v and the content per unit dose of hydrochloride Ambroxol is between 0.27% w/v and 0.33% w/v.

12. The composition according to claim 11, wherein the one or more pharmaceutically acceptable additives are selected from the group consisting of: neohesperidine dihydrochalcone, sucrose, glycerol, benzoic acid, citric acid, sodium metabisulfite, propylene glycol, ethyl alcohol, flavoring and/or purified water.

13. A process for preparing the pharmaceutical composition according to claim 2, wherein the process comprises mixing ambroxol or a pharmaceutically acceptable salt thereof and dropropizine, together with one or more pharmaceutically acceptable additives.

14. A process for obtaining the pharmaceutical composition according claim 6, wherein the process is performed by direct compression.

15. The pharmaceutical composition according to claim 3, for use in the treatment of diseases of the respiratory system that simultaneously occur with irritative and productive cough and wherein the inhibition of the cough reflex and a mucolytic and expectorant effect are concomitantly indicated; as a secretolytic therapy in acute and chronic bronchopulmonary diseases associated with abnormal mucus secretion and impaired mucosal transport and as a non-narcotic peripheral cough suppressant, indicated in the treatment of cough of any etiology, even in the presence of bronchospasms and respiratory insufficiency; pharyngitis, laryngitis, acute or chronic tracheitis, irritative cough.

16. The pharmaceutical composition according to claim 3, for use in the treatment of diseases of the respiratory system that concur simultaneously with irritative and productive cough and in which concomitantly indicated the inhibition of the cough reflex and a mucolytic and expectorant effect.

17. The pharmaceutical composition according to claim 3, for use in the treatment of diseases of the respiratory system that concur simultaneously with irritative and productive cough and wherein concomitantly indicated the inhibition of the cough reflex and a mucolytic and expectorant effect, in the group of patients presenting insomnia secondary to cough.

18. The pharmaceutical composition according to claim 15, further characterized in that it has fewer adverse effects than its active ingredients separately.
